(19) United States
(12) Patent Application Publication
(10) Pub. No.: US 2026/0191696 A1
(43) Pub. Date: Jul. 9, 2026

(54) NURSING PAD

(71) Applicant: Shenzhen Lute Jiacheng Supply Chain Management Co., Ltd., Shenzhen (CN)

(72) Inventors: Lan Li, Shenzhen (CN); Ting Zhou, Shenzhen (CN)

(21) Appl. No.: 19/553,462

(22) Filed: Mar. 2, 2026

(30) Foreign Application Priority Data
Dec. 16, 2025 (CN) 202522683741.0

(51) Int. Cl. A61F 13/14; A61F 13/15; A61F 13/53; A61F 13/537; B32B 3/30; B32B 5/02; B32B 5/18; B32B 27/12; B32B 27/36

(52) U.S. Cl. CPC A61F 13/141; A61F 13/53713; B32B 3/30; B32B 5/024; B32B 5/18; B32B 27/12; B32B 27/36; A61F 2013/15016; A61F 2013/530817; B32B 2266/0264; B32B 2266/06; B32B 2307/7265; B32B 2307/728; B32B 2555/00

(57) ABSTRACT

The present application discloses nursing pad comprises a wicking layer, an absorbent core layer, and a peripheral functional layer, which are sequentially stacked from a skin-facing side during wear outward. The absorbent core layer comprises at least one foam layer and at least one absorbent layer. The peripheral functional layer comprises at least a liquid-impermeable barrier layer disposed outward of the absorbent core layer. The nursing pad is configured such that an overall liquid retention capacity in a lower region thereof, corresponding to a lower portion of a breast during wear, is higher than an overall liquid retention capacity in an upper region thereof, corresponding to an upper portion of the breast.

### CROSS-REFERENCE TO RELATED APPLICATIONS

The present application claims priority to Chinese Patent Application No. 202522683741.0, filed on Dec. 16, 2025, the entire contents of which are incorporated herein by reference.

### TECHNICAL FIELD

The present application relates to the technical field of maternal and baby products, particularly to a nursing pad.

### BACKGROUND

Nursing pads are widely used personal care products for lactating women, designed to absorb unexpected milk leakage, thereby keeping clothing and skin dry and comfortable. Existing reusable nursing pads typically employ a multi-layer composite structure to balance absorption, leakage prevention, and wear comfort.

To enhance absorption capacity and prevent leakage, common improvement approaches include increasing the thickness or density of the absorbent layer, or utilizing higher-capacity absorbent materials. However, this pursuit of “absorption capacity” often directly leads to an increase in the overall thickness of the product. Excessively thick pads not only cause noticeable foreign body sensation and pressure, compromising wearing comfort, but may also create an undesirable visible outline when worn and, to some extent, affect breathability. Conversely, overly prioritizing product thinness and discretion necessitates restricting the amount or performance of the absorbent material, which in turn weakens its liquid storage capacity. This can lead to a risk of leakage due to absorption saturation during periods of high milk production or prolonged use.

Therefore, there is a need to provide a nursing pad to address the aforementioned technical problems.

### SUMMARY

In view of the deficiencies in the prior art, the present application provides a nursing pad, which aims to resolve the contradiction in the prior art wherein liquid storage capacity is difficult to balance with product thinness, lightness, and comfort.

In order to solve the above technical problem, one embodiment of this application proposes a nursing pad comprising: a wicking layer configured to face a wearer's skin; an absorbent core layer disposed outward of the wicking layer; and a peripheral functional layer disposed outward of the absorbent core layer; wherein the absorbent core layer comprises at least one foam layer and at least one absorbent layer; wherein the peripheral functional layer comprises at least a liquid-impermeable barrier layer disposed outward of the absorbent core layer; and wherein the nursing pad has a lower region configured to correspond to a lower portion of a breast during wear and an upper region configured to correspond to an upper portion of the breast, and wherein an overall liquid retention capacity in the lower region is higher than an overall liquid retention capacity in the upper region.

In one embodiment, the peripheral functional layer further comprises a cushioning layer laminated outward of the liquid-impermeable barrier layer.

In one embodiment, the higher overall liquid retention capacity in the lower region is provided by at least one additional absorbent member disposed within the lower region.

In one embodiment, the at least one additional absorbent member is disposed between the liquid-impermeable barrier layer and the absorbent core layer.

In one embodiment, the at least one additional absorbent member is disposed outward of the liquid-impermeable barrier layer.

In one embodiment, the foam layer comprises a fully open-cell polyester foam.

In one embodiment, a skin-facing surface of the nursing pad comprises a plurality of spaced-apart protrusions.

In one embodiment, the cushioning layer comprises a semi-closed-cell polyester foam.

In one embodiment, the at least one absorbent layer has a greater thickness in the lower region than in the upper region such that the overall liquid retention capacity in the lower region is higher.

In one embodiment, the at least one absorbent layer has a greater absorption rate per unit volume in the lower region than in the upper region such that the overall liquid retention capacity in the lower region is higher.

In one embodiment, the at least one absorbent layer comprises a higher proportion of hydrophilic fibers in the lower region than in the upper region.

In one embodiment, the at least one absorbent layer comprises a terry cloth, and wherein the terry cloth has a greater loop density or a greater loop height in the lower region than in the upper region.

Another embodiment provided by the present application is: a nursing pad comprising: a wicking layer configured to face a wearer's skin; an absorbent core layer disposed outward of the wicking layer; and a peripheral functional layer disposed outward of the absorbent core layer; wherein the absorbent core layer comprises at least one foam layer and at least one absorbent layer; wherein the peripheral functional layer comprises at least a liquid-impermeable barrier layer disposed outward of the absorbent core layer; and wherein the foam layer comprises a fully open-cell polyester foam.

In one embodiment, the peripheral functional layer further comprises a cushioning layer laminated outward of the liquid-impermeable barrier layer.

In one embodiment, the cushioning layer comprises a semi-closed-cell polyester foam.

Yet another embodiment provided by the present application is: A nursing pad comprising: a wicking layer configured to face a wearer's skin; an absorbent core layer disposed outward of the wicking layer; and a peripheral functional layer disposed outward of the absorbent core layer; wherein the absorbent core layer comprises at least one foam layer and at least one absorbent layer; wherein the peripheral functional layer comprises at least a liquid-impermeable barrier layer disposed outward of the absorbent core layer; and wherein a skin-facing surface of the nursing pad comprises a plurality of spaced-apart protrusions.

In one embodiment, the support panel is joined to the breast pump functional layer at least at a junction of a neckline edge and an underarm edge of the breast pump functional layer.

In one embodiment, the skin-facing surface defines a recess at a position configured to align with a wearer's nipple.

In one embodiment, the wicking layer comprises a unidirectional moisture transport fabric having an antimicrobial property.

In one embodiment, the wicking layer comprises fibers containing an antimicrobial additive.

The nursing pad according to the embodiments of the present application comprises a wicking layer, an absorbent core layer, and a peripheral functional layer, which are sequentially stacked from a skin-facing side during wear outward. The absorbent core layer comprises at least one foam layer and at least one absorbent layer. The peripheral functional layer comprises at least a liquid-impermeable barrier layer disposed outward of the absorbent core layer. The nursing pad is configured such that an overall liquid retention capacity in a lower region thereof, corresponding to a lower portion of a breast during wear, is higher than an overall liquid retention capacity in an upper region thereof, corresponding to an upper portion of the breast. By providing a regional structure with differentiated liquid retention capacities, the present application enables the nursing pad to effectively increase its overall effective liquid storage capacity and leakage prevention reliability without significantly increasing its overall thickness, thereby resolving the contradiction in the prior art wherein liquid storage capacity is difficult to balance with product thinness, lightness, and comfort.

### BRIEF DESCRIPTION OF THE DRAWINGS

To further clarify the technical solutions in the embodiments of this application or the prior art, a brief introduction to the drawings that need to be used in the description of the embodiments or prior art will be provided below. It is obvious that the drawings described below are merely some embodiments of this application, and for those skilled in the art, additional drawings may be obtained based on the structures shown in these drawings without requiring inventive effort.

FIG. 1 is a schematic perspective view of a nursing pad according to an embodiment of the present application;

FIG. 2 is a schematic layered structural view of a nursing pad according to a first embodiment of the present application;

FIG. 3 is a schematic layered structural view of a nursing pad according to a second embodiment of the present application;

FIG. 4 is a schematic layered structural view of a nursing pad according to a third embodiment of the present application.

The realization, functional characteristics, and advantages of the objectives of this application will be further explained in combination with the embodiments, with reference to the drawings.

### DETAILED DESCRIPTION OF THE EMBODIMENTS

In this application, the terms “provided,” “disposed,” and “connected” should be broadly understood. For example, they can refer to fixed connections, detachable connections, or integrated structures. They may indicate mechanical connections or electrical connections, direct connections, or indirect connections through an intermediary, or they may refer to internal communication between two devices, components, or parts. A person skilled in the art can understand the specific meaning of these terms in this application according to the context.

The terms “center,” “longitudinal,” “transverse,” “length,” “width,” “thickness,” “top,” “bottom,” “front,” “back,” “left,” “right,” “vertical,” “horizontal,” “inner,” “outer,” “radial,” and “circumferential” indicate directional or positional relationships based on the orientation or positional relationships shown in the figures. These terms are used solely to simplify and clarify the description of the present application and should not be interpreted as limiting the invention to a specific orientation, construction, or operation.

Additionally, the terms “first” and “second” are used only for descriptive purposes and should not be understood as indicating relative importance or implying the number of features. Therefore, a “first” or “second” feature may explicitly or implicitly include one or more of that feature. In the description of this application, “multiple” means at least two, such as two or three, unless otherwise explicitly specified.

Moreover, the above terms, in addition to indicating directional or positional relationships, may also have other meanings. For example, the term “top” may also refer to attachment or connection relationships. A person skilled in the art can understand these terms according to the specific context.

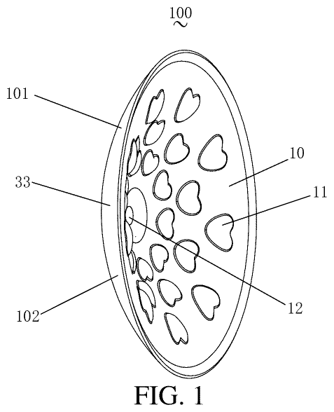

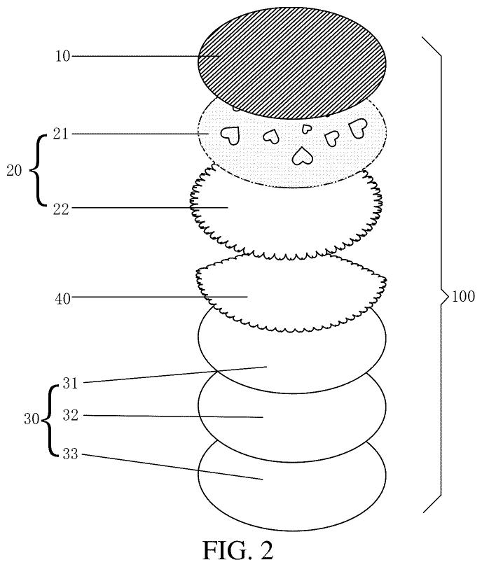

Referring to FIG. 1 and FIG. 2, FIG. 1 is a schematic perspective view of a nursing pad according to an embodiment of the present application; FIG. 2 is a schematic layered structural view of a nursing pad according to a first embodiment of the present application.

The nursing pad 100 according to an embodiment of the present application comprises a wicking layer 10, an absorbent core layer 20, and a peripheral functional layer 30, which are sequentially stacked from a skin-facing side during wear outward.

In one implementation, a skin-facing surface of the nursing pad 100 comprises a plurality of spaced-apart protrusions 11. In one implementation, the wicking layer 10 is a unidirectional moisture transport fabric having an antimicrobial property. The antimicrobial property may be achieved by incorporating a silver-ion antimicrobial masterbatch into the yarn. As the layer in direct contact with the skin, the primary function of the wicking layer 10 is to rapidly capture and directionally transport liquid.

In one implementation, the wicking layer 10 may be made of a fabric with unidirectional moisture transport functionality, for example, a knitted fabric woven from polyester fibers with modified cross-sections. The skin-facing surface of the nursing pad 100 may further comprise an array of circular or heart-shaped breathable protrusions 11. The protrusions 11 may also take other shapes to reduce the complete contact area with the skin and enhance air circulation.

In one implementation, the unidirectional moisture transport property of the wicking layer 10 can be specifically achieved by spinning synthetic fibers (e.g., polyester, nylon) with non-circular cross-sections into yarns and weaving them into fabric. The fiber cross-sections are designed to be cross-shaped, C-shaped, H-shaped, or to have multiple grooves. These grooves form continuous, nano-to micro-scale capillary channels on the fiber surfaces. Through precise control of spinning and weaving processes, the fiber grooves constituting the inner side of the fabric can be designed to have a smaller radius of curvature, generating stronger capillary adsorption force to rapidly capture liquid droplets from the skin surface. Conversely, the fiber grooves constituting the outer side of the fabric are designed to have a larger radius of curvature or a different geometry, resulting in relatively weaker capillary force. Consequently, a capillary pressure gradient from the inner side to the outer side is established across the fabric cross-section, driving the continuous unidirectional movement of liquid.

In another implementation, the wicking layer 10 may adopt a double-layer fabric structure. The inner layer is a lightweight mesh fabric woven from hydrophobic fibers (e.g., water-repellent treated polyester), and the outer layer is a dense fabric woven from strongly hydrophilic fibers (e.g., hydrophilically modified polyester, cotton, or viscose). These two layers are then tightly bonded through knitting or a composite process. The wicking layer 10 can also utilize bicomponent composite fibers (e.g., sheath-core structure, side-by-side structure). For instance, the “sheath” is a hydrophobic polymer (e.g., polypropylene), and the “core” is a hydrophilic polymer (e.g., modified polyester). Directional moisture transport is achieved through the material difference within the cross-section of a single fiber. The hydrophobic portion on the inner side “repels” liquid, forcing droplets to move outward; the hydrophilic portion on the outer side provides a strong “suction” effect, firmly adsorbing and spreading the liquid. The two work in synergy, creating a “push-pull” effect.

The wicking layer 10 is a functional fabric configured to achieve unidirectional moisture transport. As a preferred specific implementation, the fabric is woven from polyester fibers with modified cross-sections. The modified cross-sections are cross-shaped or Y-shaped, utilizing the capillary pressure differential generated by the micro-grooves formed on the fiber surfaces to achieve unidirectional liquid transport from the inside to the outside. As an alternative implementation, the wicking layer 10 is a fabric with a composite structure featuring a hydrophobic inner side and a hydrophilic outer side, for example, manufactured through double-layer weaving or differential single-side finishing processes.

To achieve the antimicrobial function, the wicking layer 10 is manufactured using one of the following methods: (a) woven from fibers containing an antimicrobial masterbatch, wherein the antimicrobial masterbatch comprises silver-ion compounds or quaternary ammonium salt-based antimicrobial agents; or (b) treated with a durable antimicrobial finishing process, wherein the finishing agent comprises microencapsulated silver ions or silicone quaternary ammonium salts that form cross-links with the fibers. The antimicrobial function ensures that the antibacterial rates of the wicking layer 10 against Staphylococcus aureus and Escherichia coli (tested according to standards such as GB/T 20944.3) remain at a high level (e.g., >70%) even after multiple washings.

The absorbent core layer 20 comprises at least one foam layer 21 and at least one absorbent layer 22. In one implementation, the foam layer 21 may be a fully open-cell polyester foam layer 21; and the absorbent layer 22 may be a terry cloth layer 22. The fully open-cell polyester foam layer 21 is a flexible foam made from polyurethane (polyester-based) material, characterized by its internal cell structure. Unlike common closed-cell or semi-closed-cell foams, the majority of its cell walls are ruptured or open, causing countless tiny cells to interconnect and form a three-dimensional, continuous, reticulated porous structure. This structure resembles a microscopic “honeycomb network,” providing continuous, low-resistance channels for the passage of liquid and air. Its primary function is not simply to “absorb” and lock in liquid, but to act as a high-speed liquid distribution network. When liquid enters from one side, it can instantly diffuse rapidly in all directions (including in-plane and through-thickness directions) within the foam via these interconnected channels by capillary action. This achieves immediate liquid reception and efficient transfer, performing uniform “pre-distribution” for the subsequent absorbent layer 22. This is a unique function unattainable by closed-cell or ordinary foams and contributes to a quick-drying capability.

The peripheral functional layer 30 comprises at least a liquid-impermeable barrier layer 31 disposed outward of the absorbent core layer 20. The liquid-impermeable barrier layer 31 is a waterproof and moisture-permeable functional layer. In one specific implementation, it is constructed by laminating a polyurethane (TPU) microporous film onto a carrier non-woven fabric. This film features a microporous structure that allows water vapor to pass through while blocking liquids. The liquid-impermeable barrier layer 31 is disposed between the absorbent core layer 20 and the cushioning sponge layer 32.

In one implementation, the peripheral functional layer 30 further comprises a cushioning sponge layer 32 laminated outward of the liquid-impermeable barrier layer 31.

The nursing pad 100 is configured such that an overall liquid retention capacity in a lower region 102, corresponding to a lower portion of a breast during wear, is higher than an overall liquid retention capacity in an upper region 101, corresponding to an upper portion of the breast. In one implementation, the lower region 102 occupies approximately three-quarters of the total coverage area of the nursing pad 100, and the upper region 101 occupies approximately one-quarter of the total coverage area of the nursing pad 100.

In one implementation, the higher overall liquid retention capacity in the lower region 102 is achieved by providing at least one additional absorbent member 40 within the lower region 102. In one implementation, the absorbent member 40 is a terry cloth.

In another implementation, the nursing pad 100 may not include an additional absorbent member 40. Instead, the absorbent layer 22 has a greater thickness in the lower region 102 than in the upper region 101, such that the overall liquid retention capacity in the lower region is higher.

In some implementations, a combination of the above two approaches may be used to achieve the higher overall liquid retention capacity in the lower region 102 compared to the upper region 101.

In some implementations, the nursing pad 100 may not include an additional absorbent member 40, and the difference in liquid retention capacity between the upper region 101 and the lower region 102 is not achieved through a thickness difference. Instead, the lower region 102 and the upper region 101 utilize different materials. For example, the absorbent layer 22 has a greater water absorption rate per unit volume in the lower region 102 than in the upper region 101. As a specific example, the absorbent layer 22 is a terry cloth, wherein the lower region 102 is locally treated with a hydrophilic finishing agent, while the upper region 101 is not treated with a hydrophilic finishing agent. Alternatively, the absorbent layer 22 in the lower region 102 is woven from yarn containing a higher proportion of hydrophilic fibers, meaning the proportion of hydrophilic fibers in the absorbent layer 22 in the lower region 102 is higher than that in the upper region 101. As another alternative, the absorbent layer 22 has a greater loop density or loop height in the lower region 102 than in the upper region 101, thereby providing superior water absorption performance in the lower region 101.

In one implementation, the additional absorbent member 40 is disposed outward of the liquid-impermeable barrier layer 31. For example, the additional absorbent member 40 may be disposed between the liquid-impermeable barrier layer 31 and the cushioning sponge layer 32; or the additional absorbent member 40 may be disposed between the liquid-impermeable barrier layer 31 and the absorbent core layer 20. The placement of the additional absorbent member 40 provides two key pathways for achieving regionally differentiated enhancement: when disposed between the liquid-impermeable barrier layer 31 and the cushioning sponge layer 32, it primarily acts as a secondary safeguard located outward of the liquid-impermeable barrier layer 31, focusing on intercepting minimal liquid that may seep through to provide final leakage redundancy; when disposed between the liquid-impermeable barrier layer 31 and the absorbent core layer 20, it actively enhances the core liquid storage capacity and absorption speed of the lower region from the inner side.

In one implementation, the cushioning sponge layer 32 is a semi-closed-cell polyester foam layer 32. The semi-closed-cell polyester foam layer 32 is a flexible foam material made from polyester-based polyurethane, characterized by an internal cell structure intermediate between “fully open-cell” and “fully closed-cell”: while most cells are interconnected, a significant portion remain isolated or semi-isolated. This structure grants it both the breathability and resilience associated with some open-cell structures, and the lightweight, softness, and good deformation recovery typical of partially closed-cell structures. Within the specific construction of the nursing pad 100, the primary function of the cushioning sponge layer 32 is not to absorb large amounts of liquid (which is the function of the fully open-cell foam layer and the terry cloth layer), but rather to serve the following three key functions:

Providing Physical Cushioning and Support: Its soft and elastic nature effectively distributes and cushions the pressure exerted by the breast on the pad, significantly enhancing wearing comfort by preventing a hard feel, while also imparting a full, rounded three-dimensional shape to the pad.

Enabling Functional Zoning and Structural Isolation: It isolates the inner functional layers responsible for core absorption and waterproofing (the wicking layer 10, absorbent core layer 20, and barrier layer) from the outermost aesthetic fabric layer. This isolation protects the delicate inner waterproof layer (particularly the microporous film) from direct exposure to high temperature and pressure during subsequent processes such as thermoforming, ensuring the integrity and reliability of the waterproof function.

Optimizing Appearance and Tactile Feel: As a filler layer beneath the outer fabric, it contributes to a smoother, more structured final product appearance, avoiding potential wrinkles or irregular contours that could arise from direct exposure of the internal laminated structure, thereby providing an improved look and feel.

In one implementation, the peripheral functional layer 30 further comprises an outer cover 33 laminated outward of the cushioning sponge layer 32. The material of the outer cover 33 may be a nylon-spandex blend fabric or a polyester-spandex blend fabric.

In one implementation, the nursing pad 100 is provided with a recess 12 at a position corresponding to a wearer's nipple during wear. In one implementation, the laminated structure of the nursing pad 100 is integrally formed with an inward-facing recess 12 in the region corresponding to the wearer's nipple via a thermoforming process. Specifically, the recess 12 is formed by selectively compressing and setting the foam layer 21 within the absorbent core layer 20 in that region during the thermoforming process using a mold with a specific protrusion. Consequently, the recess 12 presents as a smooth concave structure originating from the surface of the innermost wicking layer 10 and supported by the compressed and set foam layer 21. This structure provides a non-compressive accommodation space for the nipple, optimizing comfort and breathability, while also ensuring the structural integrity of the pad in that area and facilitating the smooth transport of milk away from the region. The protrusion structures 11 are formed on the foam layer 21 through thermoforming and are visible on the side of the nursing pad 100 that faces the wearer's body.

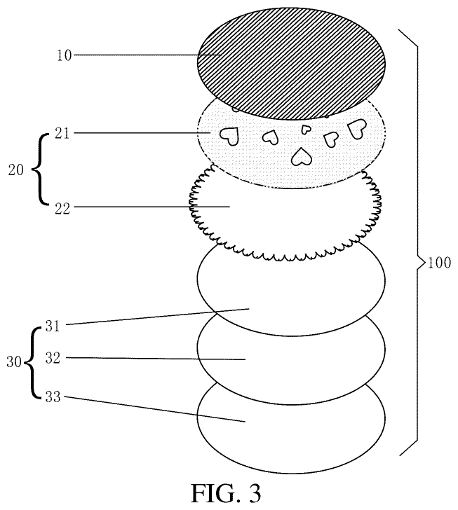

Referring to FIG. 3, which is a schematic layered structural view of a nursing pad according to a second embodiment of the present application. The difference from the first embodiment lies in that the nursing pad 100 does not include an additional absorbent member.

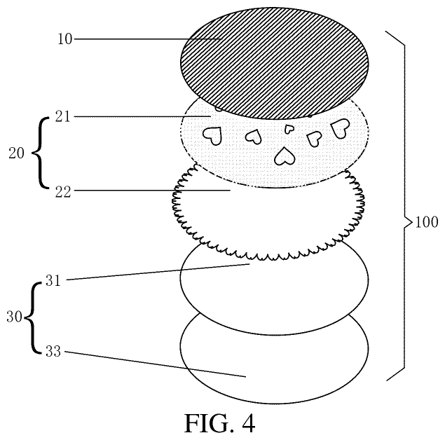

Referring to FIG. 4, which is a schematic layered structural view of a nursing pad according to a third embodiment of the present application. The difference from the first embodiment lies in that the peripheral functional layer 30 does not include a cushioning sponge layer, and the nursing pad does not include an additional absorbent member. The peripheral functional layer 30 of the third embodiment comprises a liquid-impermeable barrier layer 31 disposed outward of the absorbent core layer 20 and an outer cover 33 disposed outward of the liquid-impermeable barrier layer 31.

The nursing pad according to the embodiments of the present application comprises a wicking layer, an absorbent core layer, and a peripheral functional layer, which are sequentially stacked from a skin-facing side during wear outward. The absorbent core layer comprises at least one foam layer and at least one absorbent layer. The peripheral functional layer comprises at least a liquid-impermeable barrier layer disposed outward of the absorbent core layer. The nursing pad is configured such that an overall liquid retention capacity in a lower region thereof, corresponding to a lower portion of a breast during wear, is higher than an overall liquid retention capacity in an upper region thereof, corresponding to an upper portion of the breast. By providing a regional structure with differentiated liquid retention capacities, the present application enables the nursing pad to effectively increase its overall effective liquid storage capacity and leakage prevention reliability without significantly increasing its overall thickness, thereby resolving the contradiction in the prior art wherein liquid storage capacity is difficult to balance with product thinness, lightness, and comfort.

While certain features of the embodiments of the subject matter claimed have been illustrated and described herein, many modifications, substitutions, changes, and equivalents will now occur to those skilled in the art. Moreover, although several functional modules and their relationships have been described in detail, it is believed by those skilled in the art that some operations can be performed without using other functional modules, or other functions or relationships between functions can be established while still conforming to the subject matter claimed. Therefore, it is intended that the appended claims encompass all such modifications and variations that fall within the true spirit and scope of the embodiments of the claimed subject matter.

The above description only provides specific embodiments of the present application. It should be noted that for ordinary technicians in the technical field, without departing from the principles of the present application, several improvements and embellishments can also be made, which should also be regarded as falling within the protection scope of the present application.

What is claimed is:

1. A nursing pad comprising:
a wicking layer configured to face a wearer's skin;
an absorbent core layer disposed outward of the wicking layer; and
a peripheral functional layer disposed outward of the absorbent core layer;
wherein the absorbent core layer comprises at least one foam layer and at least one absorbent layer;
wherein the peripheral functional layer comprises at least a liquid-impermeable barrier layer disposed outward of the absorbent core layer; and
wherein the nursing pad has a lower region configured to correspond to a lower portion of a breast during wear and an upper region configured to correspond to an upper portion of the breast, and wherein an overall liquid retention capacity in the lower region is higher than an overall liquid retention capacity in the upper region.

2. The nursing pad of claim 1, wherein the peripheral functional layer further comprises a cushioning layer laminated outward of the liquid-impermeable barrier layer.

3. The nursing pad of claim 1, wherein the higher overall liquid retention capacity in the lower region is provided by at least one additional absorbent member disposed within the lower region.

4. The nursing pad of claim 3, wherein the at least one additional absorbent member is disposed between the liquid-impermeable barrier layer and the absorbent core layer.

5. The nursing pad of claim 3, wherein the at least one additional absorbent member is disposed outward of the liquid-impermeable barrier layer.

6. The nursing pad of claim 1, wherein the foam layer comprises a fully open-cell polyester foam.

7. The nursing pad of claim 1, wherein the at least one absorbent layer comprises a terry cloth layer.

8. The nursing pad of claim 2, wherein the cushioning layer comprises a semi-closed-cell polyester foam.

9. The nursing pad of claim 1, wherein a skin-facing surface of the nursing pad comprises a plurality of spaced-apart protrusions.

10. The nursing pad of claim 1, wherein the at least one absorbent layer has a greater thickness in the lower region than in the upper region such that the overall liquid retention capacity in the lower region is higher.

11. The nursing pad of claim 1, wherein the at least one absorbent layer has a greater absorption rate per unit volume in the lower region than in the upper region such that the overall liquid retention capacity in the lower region is higher.

12. The nursing pad of claim 11, wherein the at least one absorbent layer comprises a higher proportion of hydrophilic fibers in the lower region than in the upper region.

13. The nursing pad of claim 11, wherein the at least one absorbent layer comprises a terry cloth, and wherein the terry cloth has a greater loop density or a greater loop height in the lower region than in the upper region.

14. A nursing pad comprising:
a wicking layer configured to face a wearer's skin;
an absorbent core layer disposed outward of the wicking layer; and
a peripheral functional layer disposed outward of the absorbent core layer;
wherein the absorbent core layer comprises at least one foam layer and at least one absorbent layer;
wherein the peripheral functional layer comprises at least a liquid-impermeable barrier layer disposed outward of the absorbent core layer; and
wherein the foam layer comprises a fully open-cell polyester foam.

15. The nursing pad of claim 14, wherein the peripheral functional layer further comprises a cushioning layer laminated outward of the liquid-impermeable barrier layer.

16. The nursing pad of claim 15, wherein the cushioning layer comprises a semi-closed-cell polyester foam.

17. A nursing pad comprising:
a wicking layer configured to face a wearer's skin;
an absorbent core layer disposed outward of the wicking layer; and
a peripheral functional layer disposed outward of the absorbent core layer;
wherein the absorbent core layer comprises at least one foam layer and at least one absorbent layer;
wherein the peripheral functional layer comprises at least a liquid-impermeable barrier layer disposed outward of the absorbent core layer; and
wherein a skin-facing surface of the nursing pad comprises a plurality of spaced-apart protrusions.

18. The nursing pad of claim 17, wherein the skin-facing surface defines a recess at a position configured to align with a wearer's nipple.

19. The nursing pad of claim 17, wherein the wicking layer comprises a unidirectional moisture transport fabric having an antimicrobial property.

20. The nursing pad of claim 17, wherein the wicking layer comprises fibers containing an antimicrobial additive.
